(19) United States
(12) Patent Application Publication
(10) Pub. No.: US 2017/0073735 A1
(43) Pub. Date: Mar. 16, 2017

(54) SYSTEMS, METHODS AND SOFTWARE FOR RANKING POTENTIAL GEROPROTECTIVE DRUGS

(71) Applicants: Susan Eve LIFSHITZ, Jerusalem (IL); PATHWAY PHARMACEUTICALS LTD, SAR, Hong Kong (CN)

(72) Inventors: ANTON BUZDIN, ISTRA (RU); NICOLAY BORISOV, KRASNOZNAMENSK (RU); ALEXANDER ZHAVORONKOV, MOSCOW (RU)

(21) Appl. No.: 15/125,177

(22) PCT Filed: Mar. 2, 2015
(86) PCT No.: PCT/IL2015/000012

§ 371 Date: Sep. 11, 2016

Related U.S. Application Data
(60) Provisional application No. 61/950,231, filed on Mar. 10, 2014.

(51) Int. Cl. C12Q 1/68; G06F 19/12; G06F 19/26

(52) U.S. Cl. CPC C12Q 1/6809; G06F 19/26; G06F 19/12

(57) ABSTRACT

The present invention provides improved systems, methods and software for determining a pathway activation strength in old subjects relative to young subjects of the same species, the method including collecting young subject transcriptome data and old subject transcriptome data for one species to evaluate pathway activation strength (PAS) and down-regulation strength for a plurality of biological pathways, mapping the plurality of biological pathways for the activation strength and down-regulation strength from old subject samples relative to young subject samples to form a pathway cloud map and providing a gero-protective rating for each of a plurality of drugs in accordance with a drug rating for minimizing signaling pathway cloud disturbance (SPCD) in the pathway cloud map of the one species to provide a ranking of the gero-protective drugs.

### FIELD OF THE INVENTION

The present invention relates generally to gero-protective drugs, and more specifically to systems, methods and software for ranking potential gero-protective drugs.

### BACKGROUND OF THE INVENTION

The increasing burden of the aging on the economies of the developed countries is turning the quest to increase healthy life spans from an altruistic cause into a pressing economic priority required to maintain the current standards of living and facilitate economic growth (Zhavoronkov, 2013).

While no doubt exists that aging is a complex multifactorial process with no single cause or treatment (Zhavoronkov 2011; Trindade, 2013), the issue whether aging can be classified as the disease is widely debated (Rattan S, 2013 in print). Many strategies for extending organismal life spans have been proposed including replacing cells (Rodgerson, 2011) and organs, comprehensive strategies for repairing the accumulated damage, using hormetins to activate endogenous repair processes (Gaman, 2011; Gems, 2008), modulating the aging processes through specific mutations, gene therapy (Bernardes, 2012) and small molecule drugs (Kennedy, 2103). An animal's survival strongly depends on its ability to maintain homeostasis and achieved through intracellular and intercellular communication within and among different tissues (Alcedo, 2013). Many strategies for the development and validation of drugs with geroprotective properties have been proposed to help maintain the homeostasis including drugs that act on specific targets or combinations of molecular pathways (Zhavoronkov 2012) and epigenetic drugs (Vaiserman, 2012).

Presently, none of the proposed strategies for geroprotector development provide a roadmap for rapid screening, validation and clinical deployment. No methods currently exist to predict the effects of currently available drugs on human longevity and health span in a timely manner. This is partly due to the absence of the clear panel of human biomarkers of aging to effectively run clinical trials.

Many biomarkers of aging have been proposed including telomere length (Lehmann, 2013), intracellular and extracellular aggregates, racemization of the amino acids and genetic instability. Both gene expression (Wolters, 2013) and DNA methylation profiles (Horvath, 2012, Horvath, 2013, Mendelsohn, 2013) change during aging and may be used as biomarkers of aging. Many studies analyzing transcriptomes of biopsies in a variety of diseases indicated that age and sex of the patient had significant effects on gene expression (Chowers, 2003) and that there are noticeable changes in gene expression with age in mice (Weindruch, 2002, Park, 2009), resulting in development of mouse aging gene expression databases (Zahn, 2007) and in humans (Blalock, 2003; Welle, 2003; Park, 2005; Hong, 2008; de Magalhães, J. P, 2009).

Combination of protein-protein interaction and gene expression in both flies and humans demonstrated that aging is mainly associated with a small number of biological processes, might preferentially attack key regulatory nodes that are important for network stability (Xue, 2007).

The increasing burden of the aging on the economies of the developed countries is turning the quest to increase healthy life spans from an altruistic cause into a pressing economic priority required to maintain the current standards of living and facilitate economic growth (Zhavoronkov, 2013). There is an urgent need to develop and validate interventions with geroprotective properties to increase the productive health spans of the working population and maintaining performance and avoiding loss of function (Kennedy, 2012).

Our prior work with gene expression and epigenetics of various solid tumors (Zabolotneva, 2013, Zabolotneva, 2012, Mityaev, 2010 Kuzmin, 2010) provided clues that transcription profiles of cancer cells mapped onto the signaling pathways may be used to screen for and rate the targeted drugs that regulate pathways directly and indirectly related to aging and longevity. Prior studies suggested that a combination of pathways, termed pathway cloud, instead of one element of the pathway or the whole pathway might be responsible for pathological changes in the cell (Voronkov, 2012).

There is thus an urgent need to develop and validate interventions with gero-protective properties to increase the productive health spans of the working population and maintaining performance and avoiding loss of function (Kennedy, 2012). There is a further need to develop analysis tools for predicting the efficacy of a drug as a gero-protective candidate. Furthermore, there is a need for tools for predicting the efficacy of a personalized drug as a gero-protective candidate in accordance with a specific subject's genetic profile.

### SUMMARY OF THE INVENTION

It is an object of some aspects of the present invention, to provide an aging transcriptome in which the transcribed genes in old to young people are compared to define a set first of genes which are more strongly expressed (activated) in old people relative to young people and a second set of genes (repressed) which are less strongly expressed in old people relative to young people.

In some embodiments of the present invention, improved methods and software are provided for determining a pathway activation strength in old subjects relative to young subjects of the same species. In some embodiments, the species is a vertebrate species. In some embodiments, the species is a mammalian species. In some preferred embodiments, the subject is a human species.

The generic gero-protector rating approach involves collecting the transcriptome datasets from young and old patients and normalizing the data for each cell and tissue type, evaluating the pathway activation strength (PAS) for each individual pathway (FIG. 1B) and constructing the pathway cloud (PC, FIG. 1C) and screen for drugs or combinations that minimize the signaling pathway cloud disturbance (SPCD, FIG. 1D) by acting on one or multiple elements of the pathway cloud. Drugs and combinations may be rated by their ability to return the signaling pathway activation pattern closer to that of the younger tissue samples. The predictions may be then tested both in vitro and in vivo on human cells and on model organisms such as rodents, nematodes and flies to validate the screening and rating algorithms.

The present invention provides a method for ranking gero-protective drugs, the method including collecting young subject transcriptome data and old subject transcriptome data for one species to evaluate pathway activation strength (PAS) and down-regulation strength for a plurality of biological pathways, mapping the plurality of biological pathways for the activation strength and down-regulation strength from old subject samples relative to young subject samples to form a pathway cloud map and providing a gero-protective rating for each of a plurality of drugs in accordance with a drug rating for minimizing signaling pathway cloud disturbance (SPCD) in the pathway cloud map of the one species to provide a ranking of the gero-protective drugs.

There is thus provided according to an embodiment of the present invention, a method for ranking gero-protective drugs, the method including;
a. collecting young subject transcriptome data and old subject transcriptome data for one species to evaluate pathway activation strength (PAS) and down-regulation strength for a plurality of biological pathways;
b. mapping the plurality of biological pathways for the activation strength and down-regulation strength from old subⱼect samples relative to young subject samples to form a pathway cloud map; and
c. providing a gero-protective rating for each of a plurality of drugs in accordance with a drug rating for minimizing signaling pathway cloud disturbance (SPCD) in the pathway cloud map of the one species to provide a ranking of the gero-protective drugs.

Additionally, according to an embodiment of the present invention, the pathway cloud map shows at least one upregulated/activated pathway and at least one down-regulated pathway of the old subject relative to the young subject.

Furthermore, according to an embodiment of the present invention, the pathway cloud map is based on a plurality of young subjects and a plurality of old subjects.

Further, according to an embodiment of the present invention, he species is a vertebrate species.

Yet further, according to an embodiment of the present invention, the species is a mammalian species.

Additionally, according to an embodiment of the present invention, the species is a human species.

Furthermore, according to an embodiment of the present invention, the method is performed on a plurality of ethnic groups to determine an optimized ranking of the gero-protective drugs for each ethnic group.

Importantly, according to an embodiment of the present invention, the method is performed for an individual to determine an optimized ranking of the gero-protective drugs for the individual.

Notably, according to an embodiment of the present invention, the individual is an old individual.

Additionally, according to an embodiment of the present invention, the mapping step further includes mapping each of the plurality of biological pathways for the activation strength and the down-regulation strength.

Moreover, according to an embodiment of the present invention, the biological pathways are signaling pathways.

Further, according to an embodiment of the present invention, the samples are bodily samples selected from the group consisting of a blood sample, a urine sample, a biopsy, a hair sample, a nail sample, a breathe sample, a saliva sample and a skin sample.

Yet further, according to an embodiment of the present invention, the pathway activation strength is calculated by dividing the expression levels for a gene n in the old subject samples by the gene expression levels of the young subject samples.

Additionally, according to an embodiment of the present invention, the pathway activation strength is calculated in accordance with

$$SO = \frac{{\prod }_{i = 1}^{N} {[ AGEL ]}_{i}}{{\prod }_{j = 1}^{M} {[ RGEL ]}_{j}}$$

wherein [AGEL], is an activator gene expression level and [RGEL]ⱼ is a repressor gene expression level) are expression level of activators gene [symbol] i and [symbol] j, respectively.

Yet further, according to an embodiment of the present invention, to drugs or combinations that minimize the signaling pathway cloud disturbance (SPCD).

Additionally, according to an embodiment of the present invention, the SPCD is a ratio of [AGEL]_{I}, which is the activator gene #i expression level, to [RGEL]j, which is the repressor gene #j expression level, and wherein this is calculated for activator and repressor proteins in the pathway.

There is thus provided according to another embodiment of the present invention, a computer software product, the product configured for ranking gero-protective drugs, the product including a computer-readable medium in which program instructions are stored, which instructions, when read by a computer, cause the computer to;
a. collect young subject transcriptome data and old subject transcriptome data for one species to evaluate pathway activation strength (PAS) and down-regulation strength for a plurality of biological pathways;
b. map the plurality of biological pathways for the activation strength and down-regulation strength from old subject samples relative to young subject samples to form a pathway cloud map; and
c. provide a geroprotective rating for each of a plurality of drugs in accordance with a drug rating for minimizing signaling pathway cloud disturbance (SPCD) in the pathway cloud map of the one species to provide a ranking of the gero-protective drugs.

There is thus provided according to another embodiment of the present invention, a system for ranking gero-protective drugs, the system including;
a. a processor adapted to activate a computer-readable medium in which program instructions are stored, which instructions, when read by a computer, cause the processor to;
   i. collect young subject transcriptome data and old subject transcriptome data for one species to evaluate pathway activation strength (PAS) and downregulation strength for a plurality of biological pathways;
   ii. map the plurality of biological pathways for the activation strength and downregulation strength from old subject samples relative to young subject samples to form a pathway cloud map; and
   iii. provide a geroprotective rating for each of a plurality of drugs in accordance with a drug rating for minimizing signaling pathway cloud disturbance (SPCD) in the pathway cloud map of the one species to provide a ranking of the gero-protective drugs; and
b. a memory for storing the data, the pathway cloud map, SPCD and the ranking; and
c. a display for displaying the pathway cloud map.

Additionally, according to an embodiment of the present invention, the display is adapted to show the ranking by at least one of color, line thickness and visual indicia.

There is thus provided according to another embodiment of the present invention, a method of treating an old subject, the method comprising:
a. determining a ranking of gero-protective drugs according to the methods described herein; and
b. administering to the old subject one or more drugs according to said ranking.

The old subject may be an elderly person aged over fifty, over sixty, over seventy, over eighty, over ninety or over one hundred years old.

There is thus provided according to an additional embodiment of the present invention, a pharmaceutical composition for treating an old subject, the composition comprising at least one drug ranked to have a high gero-protective rating, according to the methods described herein.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a simplified flowchart for ranking potential gero-protective drugs in the effective treatment of an individual subject, in accordance with an embodiment of the present invention;

FIG. 1B shows an equation for evaluating the pathway activation strength (PAS) for each individual pathway, in accordance with an embodiment of the present invention;

FIG. 1C is a schematic of a method for constructing a pathway cloud (PC), in accordance with an embodiment of the present invention; and

FIG. 1D is an equation of a method for screening for drugs or combinations that minimize the signaling pathway cloud disturbance (SPCD), in accordance with an embodiment of the present invention.

In all the figures similar reference numerals identify similar parts.

### DETAILED DESCRIPTION OF PREFERRED EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

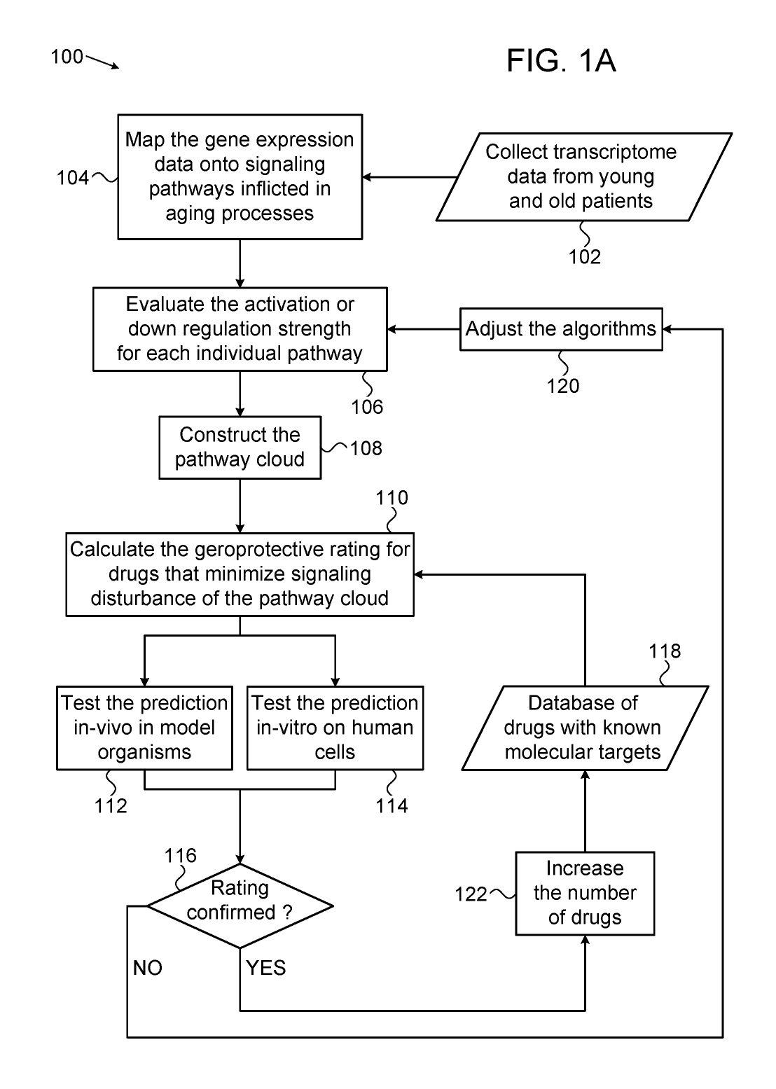

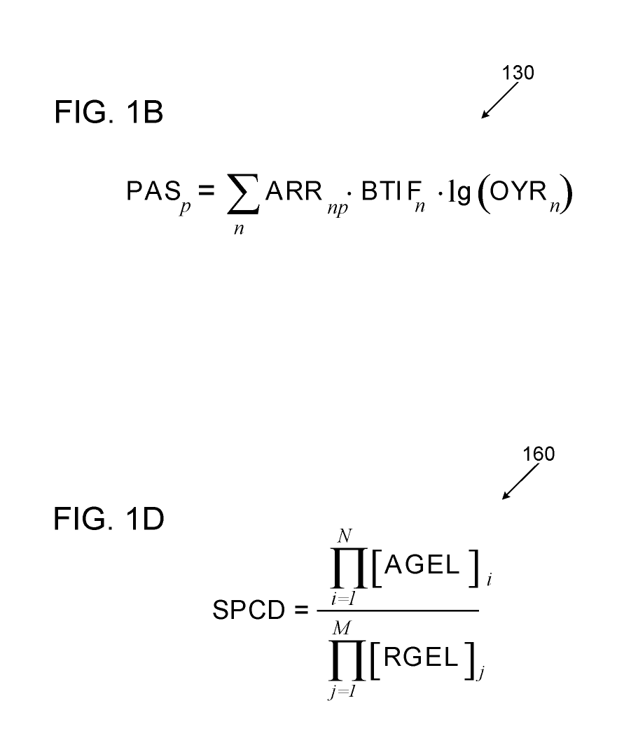

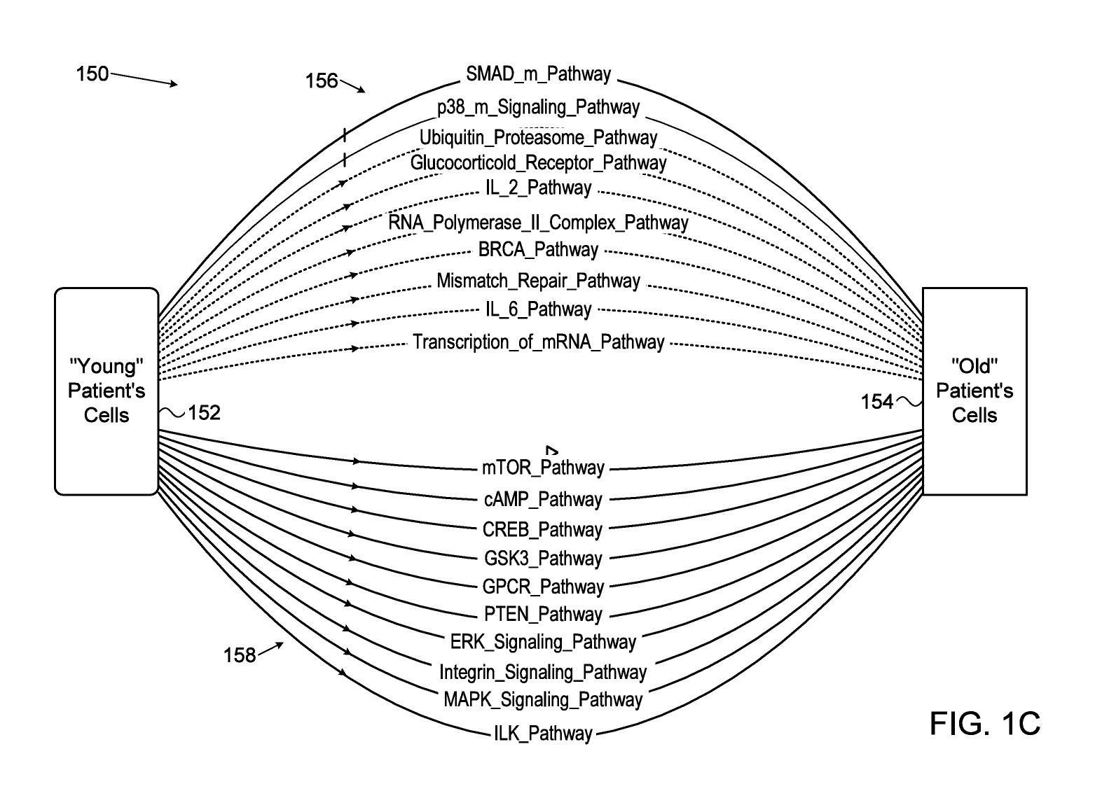

Reference is now made to FIG. 1A, which is a simplified flowchart 100 of a method for ranking potential gero-protective drugs in the effective treatment of an individual subject, in accordance with an embodiment of the present invention. Using signaling pathway cloud regulation for theoretical in-silico gero-protecor identification and ranking

In a first collecting step 102, transcriptome data from young and old patients is collected and stored in a suitable database.

In a mapping step 104, the gene expression data collected in step 102 is mapped onto signaling pathways, which are affected by aging processes.

In an evaluating step 106, the activation or down-regulation strength for each individual pathway is defined, providing one line per pathway.

Thereafter, in a cloud construction step 108, a cloud for old versus young and/or young versus old is constructed.

The lines are curved as upper halves of circles/ellipses and marked in green, for example, to denote up-regulation. Down-regulated pathways are lines curved as lower halves of circles/ellipses and marked in red, for example. Thus sets of upregulated and down-regulated paths are seen in FIG. 1C hereinbelow.

Thereafter for a number of drugs, the gero-protective rating of each drug which minimizes the signaling disturbance of the pathway cloud is determined in a gero-protective rating calculation step 110.

In one or more testing steps, 112, 114, the prediction of step 110 is tested in vivo in laboratory animals and in human species, respectively. The outputs of steps 112, 114 are testing data confirming the ratings of step 110.

A checking step 116 is performed to compare the ratings of step 110 to actual testing data.

If the testing data confirms the rating of step 110, a new drug is added to in an adding drug step 122. The data associated with the new drug is added to a database of drugs with known molecular targets in adding new drug step 118. Its potency to provide gero-protection is calculated in step 110 and it is then tested in steps 112-114. Step 114 is repeated and then, according to its results, steps 116-118 or 120, 106-114 again.

If the testing data does not match the predicted rating from step 110, the algorithm is adjusted in adjusting step 120 and steps 106-114 are repeated for that drug.

FIG. 1B shows an equation 130 of a method for evaluating the pathway activation strength (PAS) for each individual pathway, in accordance with an embodiment of the present invention.

Pathway Activation Strength (PAS) algorithm. To obtain the values of Old (case)-to-Young ratio, OYRn, one just has to divide the expression levels for a gene n in the tumor of a cancer patient by the same average value for the control healthy group. The discrete value of ARR (activator/repressor role) equals to the following numbers:

−1, when the gene/protein n is a repressor of pathway excitation;

1, if the gene/protein n is an activator of pathway excitation;

0, when the gene/protein n can be both an activator and a repressor of signal transduction;

0.5 and −0.5, respectively, if the gene/protein n is more an activator or repressor of the signaling pathway p.

The Boolean flag of BTIF (beyond tolerance interval flag) equals to zero when the CNR value lies within the tolerance limit, and to one when otherwise. During the current study, we have admitted that the CNR lies beyond the tolerance limit if it satisfies simultaneously the two criteria. First, it either higher than 3/2 or lower than 2/3, and, second, the expression level for a corresponding gene from a cancer sample of an individual patient differs by more than two standard deviations from the average expression level for the same gene from a set of analogous normal tissue samples.

FIG. 1C is a schematic of a method for constructing a pathway cloud (PC, 150), in accordance with an embodiment of the present invention.

A young person's cells' profile 152 is placed at one end of the pathways. The upregulated pathways 156 appear above an invisible horizontal axis and the down-regulated pathways 158 appear therebelow. Both the upregulated pathways 156 and down-regulated pathways 158 connect to the old person's cells' profile 154. According to some embodiments, the young person's cells' profile 152 is marked in green and the old person's cells' profile 154 is marked in red

FIG. 1D is an equation 160 of a method for screening for drugs or combinations that minimize the signaling pathway cloud disturbance (SPCD), in accordance with an embodiment of the present invention.

[AGEL]i is the activator gene #i expression level

[RGEL]j —is the repressor gene #j expression level

Here the multiplication is done over all possible activator and repressor proteins in the pathway, and [AGEL]i and [RGEL]j are gene expression levels of an activator i and repressor j, respectively.

We theorize that in order to be effective, the gero-protector or a combination of gero-protectors must regulate the pathway cloud in a way that minimizes the difference in the net differences in pathway activation or down-regulation between samples of young and old patients. Small molecules and other factors that may influence gene expression may be ranked by their ability to minimize the net difference between the pathway activation profiles of young and old cells. The algorithms for calculating the ability of the potential gero-protector to minimize signaling disturbance may be parametric and account for the effects on specific targets within signaling pathways or machine learned.

We propose a new hypothetical approach for identifying and rating the variety of factors including small molecules, proteins, stress factors and conditions with the known effects on the transcriptomes of one or more cell or tissue types or known targets (FIG. 1A). The approach may be used for general gero-protector screening, but after the validation of the algorithms in-vivo and in-vitro may be expanded to identify and predict the efficacy of personalized gero-protector regiments for individual patients based on the transcriptome information from various tissue biopsies and blood.

The generic gero-protector rating approach involves collecting the transcriptome datasets from young and old patients and normalizing the data for each cell and tissue type, evaluating the pathway activation strength (PAS) for each individual pathway (FIG. 1B) and constructing the pathway cloud (PC, FIG. 1C) and screen for drugs or combinations that minimize the signaling pathway cloud disturbance (SPCD, FIG. 1D) by acting on one or multiple elements of the pathway cloud. Drugs and combinations may be rated by their ability to return the signaling pathway activation pattern closer to that of the younger tissue samples. The predictions may be then tested both in vitro and in vivo on human cells and on model organisms such as rodents, nematodes and flies to validate the screening and rating algorithms.

Longevity studies in higher mammals take several years and decades and may cost millions of dollars. An intelligent process for predicting the activity and ranking the gero-protective activity of various factors and strengthening the prediction in rapid and cost-effective studies on cell cultures and model organisms may help increase the longevity dividend of these studies.

Gene Module Analysis is a frequently used instrument of bioinformatics. This approach makes it possible to compare and analyze in depth the experimental data obtained with transcriptome-wide analytic methods like microarray profiling and/or deep sequencing of the transcriptomes. Gene modules are gathered basing on the gene functions, on the presence of specific cis- and trans-regulatory motifs necessary for binding with proteins like transcriptional factors or different RNA molecules, or on the participation of genes in certain regulatory, biochemical or signaling pathways, including those regulating the efficiency of drug treatment of the patients.

Other popular tools for the Module Analysis are Gene Set Enrichment Analysis (GSEA) (http://www.broad.mit.edu/gsea/) and Ingenuity Pathway analysis software (http://www.ingenuity.com/). Several other databases and instruments for the pathway analysis are presented on the Tables 1 and 2.

TABLE 1
Open-access online resources enabling analytic instrumental
studies of the data of comparative and functional genomics.

| Analytic instrument | Web link |
|---|---|
| Bioconductor | http://www.bioconductor.org |
| GenePattern | http://www.broadinstitute.org/ |
| | genepattern |
| Gene Ontology | http://www.geneontology.org/ |
| | GO.tools.microarray.shtml |
| UCSC Cancer Genome Browser | https://genome-cancer.soe.ucsc.edu |
| Integrative Genomics Viewer | http://www.broadinstitute.org/igv |
| (IGV) | |
| The Cancer Genomics Pathway | http://cbioportal.org |
| Portal | |
| Gene Set Enrichment Analysis | http://www.broad.mit.edu/gsea/ |
| Ingeniuty Pathway | http://www.ingenuity.com/ |

TABLE 2
Disease and longevity genomics databases.

| | | | Type of the | |
|---|---|---|---|---|
| | | | information | |
| Database | | Type of the | in the | Access to |
| name | Web link | data¹ | database | the database |
| ICGC | http://dcc.icgc.org/ | Level I-IV | Data on the | Free |
| | | | copy number | subscription- |
| | | | variation, | based |
| | | | DNA | |
| | | | rearrangements, | |
| | | | gene | |
| | | | expression | |
| | | | and | |
| | | | mutations | |
| TCGA | http://cancergenome.nih.gov/dataportal | Level I-III | Data on the | Free |
| | | | copy number | subscription- |
| | | | variation, | based |
| | | | mRNA and | |
| | | | micro RNA | |
| | | | expression, | |
| | | | gene | |
| | | | promoter | |
| | | | methylation | |
| | | | and | |
| | | | mutations of | |
| | | | genes | |
| NCBI | http://www.ncbi.nlm.nih.gov/gap | Level I-II | Untreated | Free |
| dbGAP | | | deep | subscription- |
| | | | sequencing | based |
| | | | data | |
| COSMIC | http://www.sanger.ac.uk/genetics/CGP/cosmic | Level III-IV | Data on | Open |
| | | | somatic | |
| | | | mutations, | |
| | | | gene copy | |
| | | | number | |
| | | | variation and | |
| | | | linked | |
| | | | diseases, | |
| | | | literature | |
| | | | references | |
| Cancer | http://www.sanger.ac.uk/genetics/CGP/Census | Level IV | Data on gene | Open |
| Gene | | | mutations | |
| Census | | | | |
| WTSI CGP | http://www.sanger.ac.uk/genetics/CGP/Archive | Level I-II | Repository | Free |
| | | | of Sanger | subscription- |
| | | | sequencing | based |
| | | | data, SNP | |
| | | | data | |
| EGA | http://www.ebi.ac.uk/ega | Level I-II | Deep | Free |
| | | | sequencing | subscription- |
| | | | data | based |
| Tumorscape | http://www.broadinstitute.org/tumorscape | Level I-IV | Microarray | Open |
| | | | data browser | |
| | | | for SNP | |
| | | | analysis | |
| Oncomine | http://www.oncomine.org | Level IV | Data on gene | Password- |
| | | | expression | protected |
| | | | and copy | |
| | | | number | |
| | | | variation | |
| GEO | http://ncbi.nlm.nih.gov/geo | Level I | Data on gene | Password- |
| | | | expression | protected |
| caArray | http://caarray.nci.nih.gov | Level I | Data on gene | Password- |
| | | | expression | protected |
| UCSC | https://genome-cancer.soe.ucsc.edu | Level III-IV | Gene | Open |
| Cancer | | | expression | |
| Genome | | | and copy | |
| Browser | | | number | |
| | | | variation in | |
| | | | cancer data | |
| | | | browser | |
| The cBio | http://cbioportal.org | Level III-IV | Gene | Open |
| Cancer | | | expression | |
| Genomics | | | and copy | |
| Portal | | | number | |
| | | | variation in | |
| | | | cancer data | |
| | | | browser | |
| OMIM | http://www.ncbi.nlm.nih.gov/omim | — | Database of | Open |
| | | | inheritable | |
| | | | syndromes | |
| | | | and genes | |
| | | | having | |
| | | | phenotypic | |
| | | | manifestations | |
| | | | supplemented | |
| | | | by | |
| | | | thorough | |
| | | | thematic | |
| | | | scientific | |
| | | | literature | |
| | | | reviews | |
| Mitelman | http://cgap.nci.nih.gov/Chromosomes/Mitelman | — | Data on gene | Open |
| | | | copy number | |
| | | | variations | |
| | | | and genetic | |
| | | | translocations | |
| | | | based on | |
| | | | cytogenetic | |
| | | | studies | |

¹Type of the data: Level I - untreated data, Level II - normalized/treated data, Level III - interpreted data, Level IV - summarized data.

However, the existing published bioinformatic instruments cannot be applied efficiently for the complex quantitative analysis of transcriptome-wide data. This estimate is necessary for scoring activation and/or down-regulation of the intracellular signaling pathways in the individual tissue samples and, consequently, to conclude what biological processes may be related to the investigated physiological conditions, e.g. aging. Such an analysis may show what molecular events led to a certain physiological condition, e.g. senescence, identify a type of most effective therapeutic, to predict or follow the success the therapy and to set a prognosis for the progression of the physiological effect under investigation.

The present invention includes an algorithm of the analysis of transcriptome data for the estimation of aberrant changes in the aging cells and tissues. These changes may represent either activation or down-regulation of the signaling pathways. The transcriptome data may be obtained from either databases or from the experimental high-throughput techniques like microarray hybridization or deep sequencing of mRNA or cDNA libraries obtained for the investigated and control samples (e.g. tissue samples taken from the young healthy donors).

The starting data should contain the following information:
a) the content of gene products forming any particular signaling pathway;
b) Roles of the individual gene products in a given pathway, highlighted by a specific coefficient according to FIG. 1 and Table 3.

TABLE 3
Roles of the individual gene products in the signaling pathway

| | | | | | Contradictory |
|---|---|---|---|---|---|
| Functional | Acti- | | Rather | Rather | data or no |
| role | vator | Repressor | activator | repressor | information |
| Coefficient | 1 | −1 | 0.5 | −0.5 | 0 |

c) data on the final functional outcome of the signaling pathway activation: coefficient corresponding to the overall role of a given signaling pathway in the aging (Table 4).

TABLE 4
Functional outcomes of the individual signaling pathways in aging

| | | | Contradictory |
|---|---|---|---|
| Role in | Promotes | | data or no |
| aging | aging | Inhibits aging | information |
| Coefficient | 1 | −1 | 0 |

The data analysis can be performed using specific software enabling the following operations:

1) Identification of the differentially expressed genes. The software automatically identified differentially expressed genes in the investigated (e.g. aging) tissue sample, relatively to the control (e.g. younger) tissue samples. The software asks the user to define what sample(s) is/are control and what are the tester samples under investigation. Consequently, the statistical analysis modules, e.g. based on the pairwise t-test, or on the Bayesian criterion, enable identification of the differential genes (p<0.05), whose expression is either increased or decreased in the sample. Other cut-off p value scores may be used for the analysis, depending on the tasks of an individual study. For the sampling of normal tissues, the software also calculates mean values of the expression signal. Further calculations require the experimentally defined ratio(s)

For further calculations, we propose to use the ratio of the experimentally observed signal in the sample under investigation, to the mean value of the signal in the pool of the control samples (case-to-normal ratio, CNRn), at that this value for the non-differential genes equals 1. Non-differential genes here are the genes, for which the quantitative characteristics of gene expression belongs to the multitude of quantitative characteristics of gene expression for the normal (control) samples with the probability that exceeds a threshold value defined by the user of the technology, e.g. widely used cut-off value p=0.05.

2) Estimation of the signaling pathway activation. This estimation is performed according to the following formulae:

The overall value of the relative activation of the signaling pathway (pathway activation strength, PAS), which is used to determine the degree of apparent changes in the pathway:

PASₚ=ΣNIIₙₚ×ARRₙₚ×BTIFₙ×1(CNRₙ)

where p is the individual pathway and n is the individual gene product involved in the pathway p.

The value beyond tolerance interval flag (BTIF) is either equal to 0, when CNRn lays within the confidence interval for the controls, or equal to 1 when CNRn lays outside this confidence interval

The CNR values are calculated using the statistically treated or untreated input data obtained from the device scanning gene product expression, e.g. next generation sequencers, microarray expression scanners, and various proteome analyzing devices.

For performing this type of analysis, it is important to create and manage signaling pathway database(s), which may include the following information:

TABLE 5
Structure of the database of an individual signaling pathway

| Field | |
|---|---|
| number | Field definition |
| 1 | Identifier of a gene encoding for the protein - participant of the |
| | signaling pathway functioning |
| 2 | Name of the gene encoding for the protein - participant of the |
| | signaling pathway functioning |
| 3 | Name of the related protein or protein complex involved in the |
| | signaling pathway |
| 4 | ARR (see above) |

Among these groups of data, the following are mandatory required for performing the PAS analysis for each pathway: (i) name of the gene encoding for the protein—participant of the signaling pathway functioning, (ii) ARR.

The present invention is illustrated by the examples. The invention is not limited by these examples, but includes any additional alternative embodiments, modifications and equivalents, applicable basing on the main points of the invention.

### EXAMPLE 1

Analysis of activation levels for the Notch-signaling pathway in the aged (60-83 years old) versus younger (17-29 years old) human male bladder tissue samples.

We created a database of gene products implicated in its functioning along with their functional role reflected by the ARR value (Table 6).

TABLE 6
Database of the genes involved in functioning
of the Notch signaling pathway

| Gene | | Corresponding | |
|---|---|---|---|
| Identifier | Gene name | protein name | ARR |
| 1 | NOTCH1 | NOTCH1 | 1 |
| 2 | NOTCH2 | NOTCH2 | 1 |
| 3 | NOTCH2NL | NOTCH2NL | 1 |
| 4 | NOTCH3 | NOTCH3 | 1 |
| 5 | NOTCH4 | NOTCH4 | 1 |
| 6 | DLL1 | DLL1 | 1 |
| 7 | DLL3 | DLL3 | 1 |
| 8 | DLL4 | DLL4 | 1 |
| 9 | DTX1 | DTX1 | 1 |
| 10 | JAG1 | JAG1 | 1 |
| 11 | JAG2 | JAG2 | 1 |
| 12 | LFNG | LFNG | 1 |
| 13 | MFNG | MFNG | 1 |
| 14 | NUMB | NUMB | −1 |
| 15 | RFNG | RFNG | −1 |
| 16 | ADAM10 | ADAM10 | 1 |
| 17 | ADAM17 | CD156B | 1 |
| 18 | NCSTN | NCSTN | 1 |
| 19 | PSEN1 | PSEN1 | 1 |
| 20 | PSEN2 | PSEN2 | 1 |
| 21 | PSENEN | PEN2 | 1 |
| 22 | EP300 | EP300 | 1 |
| 23 | HDAC1 | HDAC1 | −1 |
| 24 | MAML1 | MAML1 | 1 |
| 25 | MAML2 | MAML2 | 1 |
| 26 | NCOR2 | NCOR2 | −1 |
| 27 | SNW1 | SKIIP | 1 |

The samples of human bladder were obtained from post-mortal tissue samples obtained from adult donors killed in road accidents. The samples were obtained in all cases with the written consents of the authorized persons according to EU and local ethical guidelines. Nine 17-29 y.o. (mean value −24 y.o.) bladder samples and eleven 60-83 y.o. (mean value −73 y.o.) bladder samples were obtained and further analyzed. Gene expression was investigated with the Illumina HT12 v4 gene expression microarrays (Illumina, USA). In such a way we profiled expression of the genes implicated in the functioning of the Notch pathway (Table 2). Using Student statistical criterion and a threshold p-value <0.05, we identified a list of the differential genes and calculated for them CNRn values. The set of gene expression data corresponding to the “younger” group of samples was taken as the control. These data were processed with the algorithm for the calculation of PAS, and the following Notch pathway activation data were obtained (Table 7).

TABLE 7
Pathway activation strength (PAS) scores calculated
for the aging human bladder tissue specimens

| Tissue sample | | |
|---|---|---|
| ID | PAS (Notch) | Age, y.o. |
| 1 | −15 | 60 |
| 2 | −3 | 72 |
| 3 | −29 | 75 |
| 4 | 0 | 68 |
| 5 | −4 | 66 |
| 6 | −34 | 81 |
| 7 | −19 | 83 |
| 8 | 0 | 78 |
| 9 | −6 | 76 |
| 10 | −7 | 71 |
| 11 | −21 | 78 |
| Mean | −12.5454545 | 73.45455 |
| Standard | 11.84367879 | 6.875517 |
| deviation | | |

Conclusion: we observed decreased PAS for the Notch signaling pathway in the group of older donors compared to the group of younger donors (taken as the control group).

### EXAMPLE 2

Analysis of activation levels for the ten signaling pathways in the aged (65-89 years old) versus younger (17-33 years old) human female kidney samples.

We analyzed PAS for the following signaling pathways: AHR, AKT, Circadian, DNA repair mechanisms, EGFR, ERK signaling, Estrogen pathway, FLT3, Growth hormone, Hedgehog. The information about pathway configuration and functional roles of the individual genes was taken from the public database provided by SABiosciences (SABiosciences, a Qiagen company. URL: http://www.sabiosciences.com/pathwaycentral.php (retrieved on 2013-08-13).

The samples of human kidney were obtained from post-mortal tissue samples obtained from adult donors killed in road accidents. The samples were obtained in all cases with the written consents of the authorized persons according to EU and local 2 0 ethical guidelines. Eight 17-33 y.o. (mean value −26 y.o.) bladder samples and eleven 65-89 y.o. (mean value −77 y.o.) kidney samples were obtained and further analyzed. Gene expression was investigated with the Illumina HT12 v4 gene expression microarrays (Illumina, USA). In such a way we profiled expression of the genes implicated in the functioning of the ten signaling pathways listed above. Using Student statistical criterion and a threshold p-value <0.05, we identified a list of the differential genes and calculated for them CNRn values. The set of gene expression data corresponding to the “younger” group of samples was taken as the control. These data were processed with the algorithm for the calculation of PAS, and the following pathway activation data were obtained (Table 8).

TABLE 8
Pathway activation strength (PAS) scores calculated for the aging human kidney specimens

| Tissue | Age, | | | | DNA | | | | | | |
|---|---|---|---|---|---|---|---|---|---|---|---|
| sample ID | y.o. | AHR | AKT | Circadian | repair | EGFR | ERK | Estrogen | FLT3 | GH | Hedgehog |
| 1 | 65 | 12 | −4 | 9 | −14 | −8 | −42 | −21 | 0 | −34 | 5 |
| 2 | 80 | 4 | −6 | 15 | −3 | 0 | −17 | 0 | −3 | −25 | 9 |
| 3 | 66 | 31 | −3 | 4 | −21 | 0 | −74 | −9 | 0 | −11 | 15 |
| 4 | 74 | 19 | −11 | 8 | 0 | −13 | −48 | −15 | −8 | −23 | 16 |
| 5 | 79 | 8 | −17 | 21 | −30 | −9 | −12 | −25 | −5 | −16 | 3 |
| 6 | 74 | 9 | 0 | 4 | −6 | 0 | −26 | −29 | −12 | −10 | 9 |
| 7 | 82 | 1 | −9 | 13 | −5 | −3 | −53 | −7 | 0 | −44 | 21 |
| 8 | 79 | 34 | −6 | 8 | 1 | 0 | −30 | −37 | 0 | −7 | 6 |
| 9 | 76 | 0 | −7 | 7 | 0 | −14 | −17 | 0 | −7 | −39 | 8 |
| 10 | 89 | 14 | −12 | 8 | −12 | −1 | −23 | −8 | −6 | −27 | 11 |
| 11 | 79 | 22 | 0 | 12 | −11 | 0 | −36 | −26 | −9 | −14 | 3 |
| Mean | 76.636 | 14 | −6.818 | 9.909 | −9.181 | −4.364 | −34 | −16.090 | −4.5 | −22.7 | 9.636 |
| Standard | 6.874 | 11.437 | 5.193 | 5.029 | 9.724 | 5.572 | 18.67 | 12.340 | 4.251 | 12.39 | 5.714 |
| deviation | | | | | | | | | | | |

Conclusion: The analysis enabled us to identify upregulated and downregulated signaling pathways in the female aging kidney samples. ERK, Estrogen, Growth hormone, DNA repair, AKT, EGFR and FLT3 pathways appeared to be downregulated in the older female kidney tissue samples. In contrast, the pathways AHR, Circadian and Hedgehod were upregulated in the older kidney samples.

### EXAMPLE 3

Evaluation of the geroprotector activity of the chemicals on human fibroblasts.

The culture of human fibroblasts was isolated from skin of a healthy adult male donor and cultured for five passages in DMEM/F12 medium supplemented by 10% FBS. The tissue sample was obtained with the written consent of a donor according to EU and local ethical guidelines. An aliquot containing −10 million cells was frozen in liquid nitrogen (aliquot A), whereas the remaining part of the cell culture was grown for 20 additional passages in the same medium. The cells were then harvested and stored in liquid nitrogen (aliquot B). Aliquot B cells were then cultured for two additional passages and then cultured for 48 hours in the presence of four putative geroprotector drugs. The resulting cells were harvested (aliquot C). The transcriptomes of the aliquots A, B and C were investigated with the Illumina HT12 v4 gene expression microarrays (Illumina, USA). In such a way we profiled expression of the genes implicated in the functioning of the following ten signaling pathways: AHR, AKT, Circadian, DNA repair mechanisms, EGFR, ERK signaling, Estrogen pathway, FLT3, Growth hormone, Hedgehog. The information about pathway configuration and functional roles of the individual genes was taken from the public database provided by SABiosciences (SABiosciences, a Qiagen company.

Using Student statistical criterion and a threshold p-value <0.05, we identified a list of the differential genes and calculated for them CNRₙ values. The set of gene expression data corresponding to the “aliquot A” group of samples was taken as the control. The data were processed with the algorithm for the calculation of PAS, and the following pathway activation data were obtained (Table 9).

TABLE 9
Pathway activation strength (PAS) scores calculated for the human fibroblasts cultured
in the presence or in the absence of putative gero-protector chemicals.

| | Gero- | | | | DNA | | | | | | |
|---|---|---|---|---|---|---|---|---|---|---|---|
| Sample | protector | AHR | AKT | Circadian | repair | EGFR | ERK | Androgen | FLT3 | GH | Hedgehog |
| Aliquot | none | 6 | −16 | 7 | −9 | −19 | −29 | −17 | 2 | −9 | 16 |
| B | | | | | | | | | | | |
| Aliquot | none | 8 | −19 | 4 | −14 | −22 | −30 | −24 | 0 | −9 | 20 |
| C1 | | | | | | | | | | | |
| Aliquot | Spermidine | 3 | −10 | 5 | −10 | −15 | −45 | −25 | 1 | −9 | 17 |
| C2 | 1 mM | | | | | | | | | | |
| Aliquot | Spermidine | 2 | −12 | 8 | −10 | −16 | −56 | −19 | 0 | −9 | 18 |
| C3 | 1 mM | | | | | | | | | | |
| Aliquot | Wortmannin | 1 | −11 | 6 | −8 | −13 | −14 | −15 | −3 | −7 | 12 |
| C4 | 0.5 mM | | | | | | | | | | |
| Aliquot | Wortmannin | 2 | −9 | 5 | −9 | −14 | −11 | −18 | −1 | −9 | 10 |
| C5 | 0.5 mM | | | | | | | | | | |
| Aliquot | Resveratrol | 3 | −6 | 4 | −2 | −6 | −3 | −7 | 2 | −5 | 9 |
| C6 | 1 mM | | | | | | | | | | |
| Aliquot | Resveratrol | 3 | −5 | 3 | −1 | −5 | −6 | −11 | 3 | −4 | 11 |
| C7 | 1 mM | | | | | | | | | | |
| Aliquot | Lonidamine | 2 | −7 | 5 | −5 | −5 | −8 | −4 | −1 | −11 | 17 |
| C8 | 5 mM | | | | | | | | | | |
| Aliquot | Lonidamine | 4 | −8 | 7 | −8 | −9 | −9 | −6 | −1 | −15 | 22 |
| C9 | 5 mM | | | | | | | | | | |

Zero PAS value means identical signaling pathway activation pattern in the older (aliquots B and C) and younger (aliquot A) fibroblasts. Negative and positive PAS mean, respectively, downregulated and upregulated pathway in the older fibroblasts. The present invention proposes ranking of the geroprotector drugs according to their ability to compensate aging-related differences in signaling pathway activation reflected by the PAS values.

We observed that among the four tested gero-protectors, PAS patterns were better reverted to the “younger” state by 1 mM Resveratrol (7/10 of the pathways tested). Resveratrol can be, therefore, considered as the best putative geroprotector drug in this combined experimental and biomathematical system for human skin fibroblasts.

Some embodiments of the present invention are directed to gero-protective drugs, defined in accordance with the method of maximizing at least one gero-protective characteristic, as described herein.

Some further embodiments of the present invention are directed to gero-protective drugs combinations or formulations, defined in accordance with the method of maximizing at least one gero-protective characteristic of a plurality of drugs, as described herein.

### REFERENCES:

Alcedo, J., Flatt, T., & Pasyukova, E. G. (2013). Neuronal inputs and outputs of aging and longevity. Frontiers in genetics, 4.

Blalock, E. M., Chen, K. C., Sharrow, K., Herman, J. P., Porter, N. M., Foster, T. C., & Landfield, P. W. (2003). Gene microarrays in hippocampal aging: statistical profiling identifies novel processes correlated with cognitive impairment. The Journal of neuroscience, 23(9), 3807-3819.

Bernardes de Jesus, B., Vera, E., Schneeberger, K., Tejera, A. M., Ayuso, E., Bosch, F., & Blasco, M. A. (2012). Telomerase gene therapy in adult and old mice delays aging and increases longevity without increasing cancer. EMBO molecular medicine, 4(8), 691-704.

Chowers, I., Liu, D., Farkas, R. H., Gunatilaka, T. L., Hackam, A. S., Bernstein, S. L., . . . & Zack, D. J. (2003). Gene expression variation in the adult human retina. Human molecular genetics, 12(22), 2881-2893.

de Magalhaes, J. P., Curado, J., & Church, G. M. (2009). Meta-analysis of age-related gene expression profiles identifies common signatures of aging. Bioinformatics, 25(7), 875-881.

Gaman, L., Stoian, I., & Atanasiu, V. (2011). Can ageing be slowed?: Hormetic and redox perspectives. Journal of medicine and life, 4(4), 346.

Gems, D, & Partrige, L. (2008). Stress-response hormesis and aging: “that which does not kill us makes us stronger”. Cell Metab 7, 200-3.

Hong, M. G., Myers, A. J., Magnusson, P. K., & Prince, J. A. (2008). Transcriptome-wide assessment of human brain and lymphocyte senescence. PLoS One, 3(8), e3024.

Horvath, S., Zhang, Y., Langfelder, P., Kahn, R. S., Boks, M. P., van Eijk, K., . . . & Ophoff, R. A. (2012). Aging effects on DNA methylation modules in human brain and blood tissue. Genome Biol, 13(10), R97.

Horvath, S. (2013). DNA methylation age of human tissues and cell types. Genome biology, 14(10), R115.

Kennedy, B. (2012). Gerontology: More funding for studies of ageing. Nature,487(7405), 39-39.

Brian K. Kennedy, Juniper K. Pennypacker, Drugs That Modulate Aging: The Promising yet Difficult Path Ahead, Translational Research, Available online 20 November 2013, ISSN 1931-5244, http://dx.doi.org/10.1016/j.trsl.2013.11.007.

Kuzmin D, Gogvadze E, Kholodenko R, Grzela D P, Mityaev M, Vinogradova T, Kopantzev E, Malakhova G, Suntsova M, Sokov D, Ivies Z, Buzdin A. (2010). Novel strong tissue specific promoter for gene expression in human germ cells. BMC Biotechnol. 2010 Aug 17;10:58. doi: 10.1186/1472-6750-10-58. PMID: 20716342.

Lehmann, G., Muradian, K. K., & Fraifeld, V. E. (2013). Telomere length and body temperature—independent determinants of mammalian longevity?. Frontiers in genetics, 4.

Mendelsohn, A. R., & Larrick, J. W. (2013). The DNA Methylome as a biomarker for epigenetic instability and human aging. Rejuvenation research,16(1), 74-77.

Mityaev MV, Kopantzev EP, Buzdin AA, Vinogradova TV, Sverdlov ED. (2010) Enhancer element potentially involved in human survivin gene promoter regulation in lung cancer cell lines. Biochemistry (Mosc). 2010 Feb;75(2):182-91. PMID: 20367605.

Park, S. K., Kim, K., Page, G. P., Allison, D. B., Weindruch, R., & Prolla, T. A. (2009). Gene expression profiling of aging in multiple mouse strains: identification of aging biomarkers and impact of dietary antioxidants. Aging cell,8(4), 484-495.

Park, S. K., & Prolla, T. A. (2005). Gene expression profiling studies of aging in cardiac and skeletal muscles. Cardiovascular research, 66(2), 205-212.

Rattan, S. (2013). Aging is not a disease: implications for intervention. Aging and Disease.

Rodgerson, D. O., & Harris, A. G. (2011). A comparison of stem cells for therapeutic use. Stem Cell Reviews and Reports, 7(4), 782-796.

Shostal, O. A., & Moskalev, A. A. (2012). The genetic mechanisms of the influence of the light regime on the lifespan of Drosophila melanogaster. Frontiers in genetics, 3.

Trindade, L. S., Aigaki, T., Peixoto, A. A., Balduino, A., da Cruz, I. B. M., & Heddle, J. G. (2013). A novel classification system for evolutionary aging theories. Frontiers in genetics, 4.

Vaiserman, A. M., & Pasyukova, E. G. (2012). Epigenetic drugs: a novel anti-aging strategy?. Frontiers in genetics, 3.

Voronkov, A., & Krauss, S. (2012). Wnt/beta-catenin signaling and small molecule inhibitors. Current Pharmaceutical Design, 19(4), 634.

Wolters, S., & Schumacher, B. (2013). Genome maintenance and transcription integrity in aging and disease. Frontiers in genetics, 4.

Welle, S., Brooks, A. I., Delehanty, J. M., Needler, N., & Thornton, C. A. (2003). Gene expression profile of aging in human muscle. Physiological genomics, 14(2), 149-159.

Weindruch, R., Kayo, T., Lee, C. K., & Prolla, T. A. (2002). Gene expression profiling of aging using DNA microarrays. Mechanisms of ageing and development, 123(2), 177-193.

Xue, H., Xian, B., Dong, D., Xia, K., Zhu, S., Zhang, Z., . . . & Han, J. D. J. (2007). A modular network model of aging. Molecular systems biology, 3(1).

Zabolotneva A A, Bantysh O, Suntsova M V, Efimova N, Malakhova G V, Schumann G G, Gayfullin N M, Buzdin A A. (2012). Transcriptional regulation of human-specific SVAF □ retrotransposons by cis-regulatory MAST2 sequences. Gene. 2012 Aug 15;505(1):128-36. doi: 10.1016/j.gene.2012.05.016. Epub 2012 May 15. PMID: 22609064.

Zabolotneva, A., Zhavoronkov, A. A., Shegay, P. V., Gaifullin, N. M., Alekseev, B. Y., Roumiantsev, S. A., & Buzdin, A. A. (2013). A systematic experimental evaluation of microRNA markers of human bladder cancer. Frontiers in Genetics, 4, 247.

Zabolotneva A A, Zhavoronkov A, Garazha A V, Roumiantsev S A, Buzdin A A. (2013) Characteristic patterns of microRNA expression in human bladder cancer. Front Genet. 2013 Jan 4;3:310. doi: 10.3389/fgene.2012.00310. eCollection 2012. PMID:23316212.

Zahn, J. M., Poosala, S., Owen, A. B., Ingram, D. K., Lustig, A., Carter, A., . . . & Becker, K. G. (2007). AGEMAP: a gene expression database for aging in mice. PLoS genetics, 3(11), e201.

Zhang, H. (2007). Molecular signaling and genetic pathways of senescence: its role in tumorigenesis and aging. Journal of cellular physiology, 210(3), 567-574.

Zhavoronkov, A., & Cantor, C. R. (2011). Methods for structuring scientific knowledge from many areas related to aging research. PloS one, 6(7), e22597.

Zhavoronkov, A., Smit-McBride, Z., Guinan, K. J., Litovchenko, M., & Moskalev, A. (2012). Potential therapeutic approaches for modulating expression and accumulation of defective lamin A in laminopathies and age-related diseases. Journal of Molecular Medicine, 90(12), 1361-1389.

Zhavoronkov, A., & Litovchenko, M. (2013). Biomedical Progress Rates as New Parameters for Models of Economic Growth in Developed Countries. International journal of environmental research and public health, 10(11), 5936-5952.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

1. A method for ranking gero-protective drugs, the method comprising:
a. collecting young subject transcriptome data and old subject transcriptome data for one species to evaluate pathway activation strength (PAS) and downregulation strength for a plurality of biological pathways;
b. mapping said plurality of biological pathways for said activation strength and downregulation strength from old subject samples relative to young subject samples to form a pathway cloud map; and
c. providing a geroprotective rating for each of a plurality of drugs in accordance with a drug rating for minimizing signaling pathway cloud disturbance (SPCD) in said pathway cloud map of said one species to provide a ranking of said gero-protective drugs.

2. A method according to claim 1, wherein said pathway cloud map shows at least one upregulated/activated pathway and at least one downregulated pathway of said old subject relative to said young subject.

3. A method according to claim 2, wherein said pathway cloud map is based on a plurality of young subjects and a plurality of old subjects.

4. A method according to claim 2, wherein said species is a vertebrate species.

5. A method according to claim 4, wherein said species is a mammalian species.

6. A method according to claim 5, wherein said species is a human species.

7. A method according to claim 6, wherein said method is performed on a plurality of ethnic groups to determine an optimized ranking of said gero-protective drugs for each ethnic group.

8. A method according to claim 6, wherein said method is performed for an individual to determine an optimized ranking of said gero-protective drugs for said individual.

9. A method according to claim 8, wherein said individual is an old individual.

10. A method according to claim 1, wherein said mapping step further comprises mapping each of said plurality of biological pathways for said activation strength and said downregulation strength.

11. A method according to claim 1, wherein said biological pathways are signaling pathways.

12. A method according to claim 1, wherein said samples are bodily samples selected from the group consisting of a blood sample, a urine sample, a biopsy, a hair sample, a nail sample, a breathe sample, a saliva sample and a skin sample.

13. A method according to claim 1, wherein said pathway activation strength is calculated by dividing the expression levels for a gene n in said old subject samples by said gene expression levels of said young subject samples.

14. A method according to claim 1, wherein said pathway activation strength is calculated in accordance with
$$SO = \frac{{\prod }_{i = 1}^{N} {[ AGEL ]}_{i}}{{\prod }_{j = 1}^{M} {[ RGEL ]}_{j}}$$
wherein [AGEL]ᵢ is an activator gene expression level and [RGEL]ⱼ is a repressor gene expression level) are expression level of activators gene [symbol] i and [symbol] j, respectively.

15. A method according to claim 14, wherein said SPCD is a ratio of [AGEL]_{I}, which is the activator gene #i expression level, to [RGEL]j, which is the repressor gene #j expression level, and wherein this is calculated for activator and repressor proteins in said pathway.

16. A computer software product, said product configured for ranking gero-protective drugs, the product comprising a computer-readable medium in which program instructions are stored, which instructions, when read by a computer, cause the computer to:
a. collect young subject transcriptome data and old subject transcriptome data for one species to evaluate pathway activation strength (PAS) and down-regulation strength for a plurality of biological pathways;
b. map said plurality of biological pathways for said activation strength and down-regulation strength from old subject samples relative to young subject samples to form a pathway cloud map; and
c. provide a gero-protective rating for each of a plurality of drugs in accordance with a drug rating for minimizing signaling pathway cloud disturbance (SPCD) in said pathway cloud map of said one species to provide a ranking of said gero-protective drugs.

17. A system for ranking gero-protective drugs, the system comprising:
a. a processor adapted to activate a computer-readable medium in which program instructions are stored, which instructions, when read by a computer, cause the processor to:
i. collect young subject transcriptome data and old subject transcriptome data for one species to evaluate pathway activation strength (PAS) and down-regulation strength for a plurality of biological pathways;
ii. map said plurality of biological pathways for said activation strength and down-regulation strength from old subject samples relative to young subject samples to form a pathway cloud map; and
iii. provide a geroprotective rating for each of a plurality of drugs in accordance with a drug rating for minimizing signaling pathway cloud disturbance (SPCD) in said pathway cloud map of said one species to provide a ranking of said gero-protective drugs; and
b. a memory for storing said data, said pathway cloud map, SPCD and said ranking; and
c. a display for displaying said pathway cloud map.

18. A system according to claim 17, wherein said display is adapted to show said ranking by at least one of color, line thickness and visual indicia.

19. (canceled)

20. (canceled)
